(19) United States
(12) Patent Application Publication
(10) Pub. No.: US 2003/0065368 A1
(43) Pub. Date: Apr. 3, 2003

(54) Muscle stimulator apparatus

(76) Inventor: Martin Van Der Hoeven, Del Mar, CA (US)

Correspondence Address: BROWN, MARTIN, HALLER & MCCLAIN LLP, 1660 UNION STREET, SAN DIEGO, CA, 92101-2926, US

(21) Appl. No.: 10/225,398

(22) Filed: Aug. 19, 2002

Related U.S. Application Data
(60) Provisional application No. 60/313,237, filed on Aug. 17, 2001.

(51) Int. Cl. A61N 1/18

(52) U.S. Cl. 607/48

(57) ABSTRACT

A muscle stimulator apparatus having wiring pads, corresponding to anatomical positioning of muscle groups of a user, and connected to at least one actuator is used to provide electrical stimulation to selected muscles or muscle groups. These actuators have control panels which allow the user to select different modes and intensity levels to vary the electrical stimulation administered to the muscles as desired. The electrical stimulation is varied by utilizing press buttons located on the control panel.

### CROSS-REFERENCES TO RELATED APPLICATIONS

This application claims the benefit of priority of U.S. provisional application Serial No. 60/313,237 filed Aug. 17, 2001 which is incorporated herein by reference in its entirety.

### FIELD OF THE INVENTION

The present invention relates to an apparatus that a user wears around a desired muscle group. The apparatus produces electrical current or impulses which stimulates a specific muscle group.

### BACKGROUND OF THE INVENTION

Prior muscle stimulator devices use a single actuator to produce an electric current to stimulate muscles. These prior devices have either only a set of wiring pad contact (“wiring”) configuration or portable wiring positioned without any preferred orientation anatomically. The disadvantage with the stimulators that utilize portable wiring is that the user runs the risk of injury if not properly positioned on the body and is unable to isolate a specific muscle group. The disadvantage with the single wiring configuration is that it does not allow for the simultaneous stimulation of dual muscle groups which comprises the majority of muscle groups in the body.

### SUMMARY OF THE INVENTION

It is an object of the present invention to provide a new and improved apparatus for stimulation of particular muscle groups in a human body in order to obtain greater anatomical specificity using multiple actuators.

The present invention provides a muscle stimulator apparatus capable of utilizing multiple actuators. At least two battery operated actuators are releasably connected to a belt which is placed on a desired muscle group such as the abdominal, stomach, lower back, quadriceps, thigh or buttock regions. These actuators can be set at different modes and intensities (i.e. different voltage, current and/or frequencies or patterns of frequencies) to vary the amount of electrical stimulation administered to the muscles as desired by the user.

According to one aspect of the present invention, a stimulator apparatus is provided which comprises a belt having first and second ends and top and bottom faces, at least one pair of power connectors located on the top face of the belt, at least two actuators that are releasably engaged to the power connectors, and at least one pair of anatomically positioned wiring pads on the bottom face of the belt. The actuator has a top face and a bottom face wherein the top face comprises a control panel and a battery cover. The control panel allows for different modes and intensities to be selected and performed by the user as desired. Preferably the control panel allows for a total of two different modes and ten different levels of intensity to vary the frequencies and patterns of electrical impulses that may be selected by the user as desired. The bottom face of the actuator has preferably female connectors that are used for releasably engaging the actuator to the power connectors contained on the top face of the belt. The belt is wide through the middle and long and slender on the sides to conform anatomically to the body.

In a preferred embodiment using four actuators, the power connector pairs are coupled and oriented on the top face of the belt in a diamond configuration. This diamond configuration corresponds to the anatomical positioning of the muscle groups on which the apparatus is used. The power connectors contain a male portion located on the top face of the belt that allows for releasable engagement to the actuators. A mating female portion is located on the bottom face of the actuator. An example of appropriate connectors that may be used are conventional snap connectors or any other like conductive connectors. The bottom face of the belt contains the bottom ends of the power connectors. These bottom ends are positioned adjacent to and touching the anatomically positioned wiring pads. The bottom face of the belt has at least two wiring pads that are configured so that they are coupled in order to maximize conductivity but are also anatomically positioned to obtain maximum results.

Multiple wiring pads may be used. As few as one pair to three pairs (two to six units) of anatomically positioned wiring pad configurations are preferably contained on the bottom face of the belt. A user may apply a layer of gel to the wiring pads or the desired area to be stimulated prior to securing the belt around the area. When the actuators are turned on, the electrical stimulus or impulses are conducted through the gel from wiring configurations to the user's desired muscle group. This gel is water based and is preferred to make electrical contact between the pads and the user's body.

### BRIEF DESCRIPTION OF THE DRAWINGS

The present invention will be better understood from the following detailed description of an exemplary embodiment of the invention, taken in conjunction with the accompanying drawings in which:

FIG. 1 is a rear perspective view of the belt;

FIG. 2 is a front perspective view of the belt with no actuators attached;

FIG. 3 is an enlarged view of the back face of the belt;

FIG. 3A is an enlarged view of the back face of an alternative embodiment of the belt;

FIG. 4 is a top view of the belt with the actuators attached;

FIG. 5 is a top view of the belt without the actuators attached;

FIG. 6 is a top view of the actuator; and

FIG. 7 is a bottom view of the actuator.

### DETAILED DESCRIPTION OF THE DRAWINGS

FIG. 1 is a rear perspective view of the belt 10. The belt 10 has first and second ends and a top and bottom face. This figure illustrates the view of the bottom face of the belt 10. The belt 10, made of a non-conductive material, has a wide section 20 contained through the middle section of the belt, and a slender section 22 toward the ends of the belt. Contained on opposite ends of the belt 10 are hook and loop fasteners 12 for securing the belt 10 to the desired area on a user's body.

Additionally, on the bottom face of the belt 10 are contained at least one pair of anatomically positioned wiring pads 14. These pads 14 are positioned at a distance from each other but in a coupled orientation. A water based gel is applied to the wiring pads and the wiring pads are placed against the muscles on a user's body to provide electrical stimulus or impulses to muscles. Bottom ends of power connectors 16, which are positioned adjacent to and touching the anatomically positioned wiring pads, receive an electrical current or impulse from actuators, shown in FIG. 4, which is conducted through the pads. The electrical impulses cause the targeted muscle group to contract and relax. Alternatively, the actuators and wiring pads can be contained on differently shaped belts and alternative fasteners can be used.

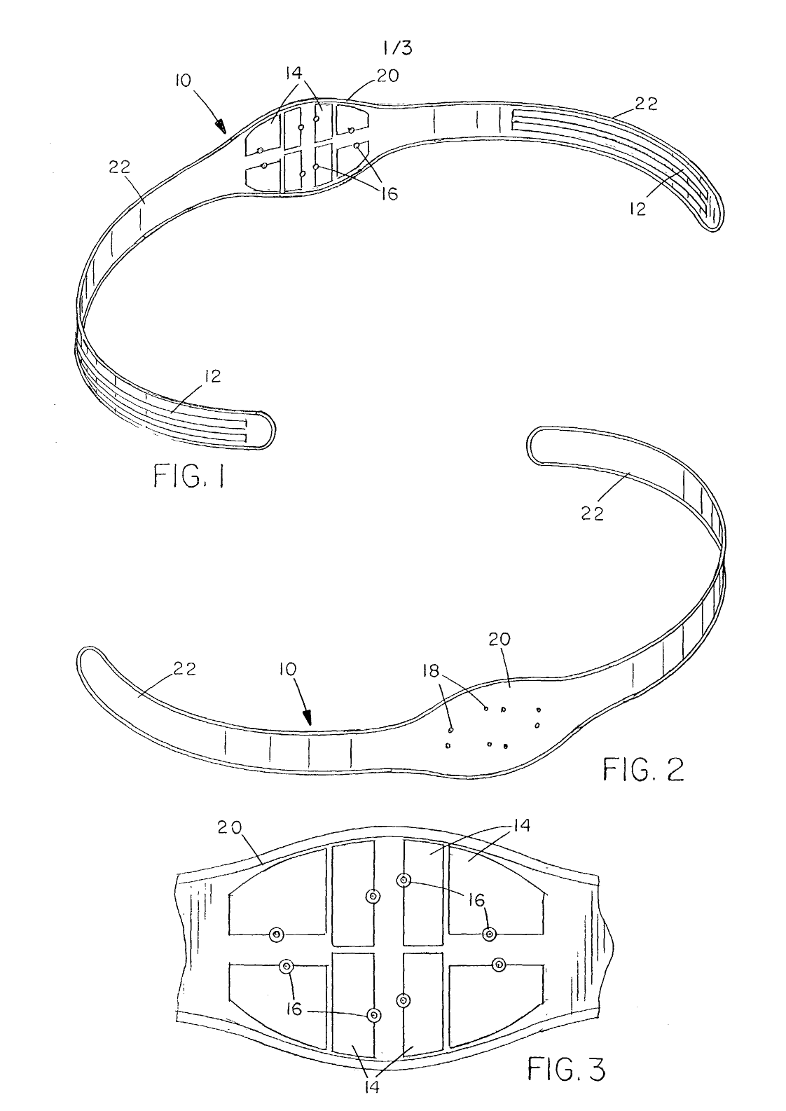

FIG. 2 is a front perspective view of the belt 10 with no actuators attached. This illustration corresponds to the top face of the belt 10. The top face of the belt 10 contains at least one pair of power connectors 18 which contain a male portion located on the top face of the belt allowing for releasable engagement to the actuators. The configuration of the power connectors 18 illustrated here is diamond shaped which correspond to the anatomical positioning of muscles groups. The configuration may alternatively be circular, square, linear, dual, triangular, or any other such shape. This configuration corresponds to the anatomical positioning of the muscles of the user. However, not all of the power connectors 18 are required if stimulation of a more focused area is desired.

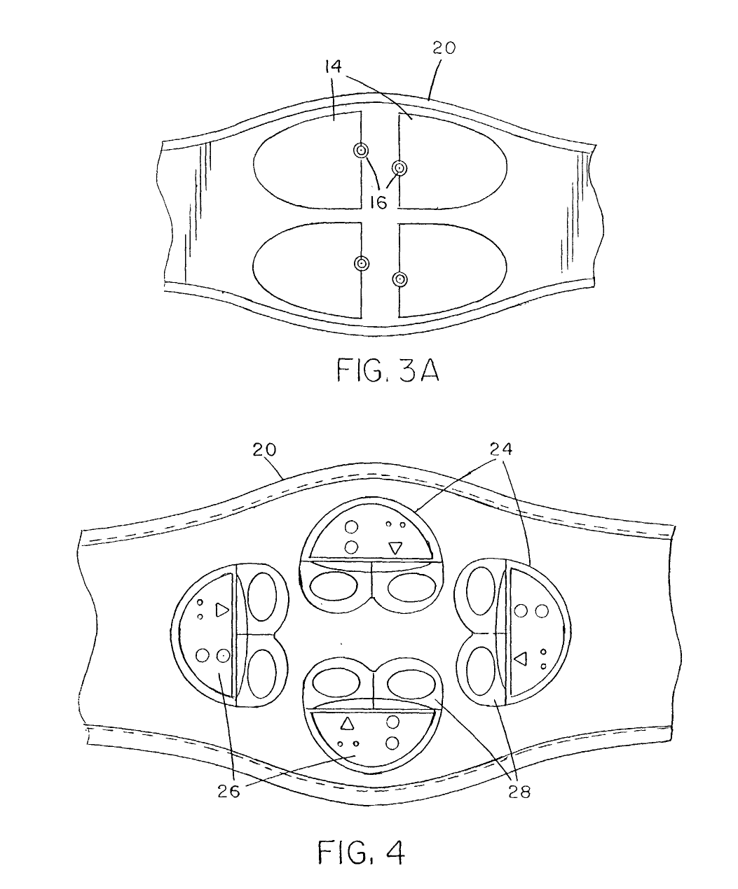

FIGS. 3 and 3A are enlarged views of the back face of the belt 10. The configuration of the pads 14 allows for application of electrical impulses over a more specific area in order to obtain optimum results. The pad size, shape, and space between the pads 14 allows the electrical impulses to remain in an isolated area of the user's body so that the specific muscle groups can be stimulated rather than merely the entire surface area where the apparatus is placed. The horizontal distance is correlated to the anatomical space on a user's back and stomach between the muscle groups. The bottom ends of the power connectors 16 can be located to target specific muscle groups. FIG. 3A illustrates differently shaped wiring pads 24 that are larger in shape but fewer in number.

FIG. 4 is a top view of the belt with the actuators 24 attached. This embodiment shows four actuators attached to the belt. The apparatus shown can accommodate between one and four actuators 24 depending on the desired results. The actuator has a top face and a bottom face wherein the top face comprises a control panel 26 and a battery cover 28. The control panel 26 allows for different modes and levels to be selected and utilized by the user as desired. Preferably the control panel 26 allows for a total of two different modes and ten different intensity levels to vary the intensity and patterns of electrical impulses that may be selected by the user as desired. The bottom face of the actuator 24 has preferably female connectors that are used for releasably engaging the actuator to the power connectors 18 contained on the top face of the belt 10.

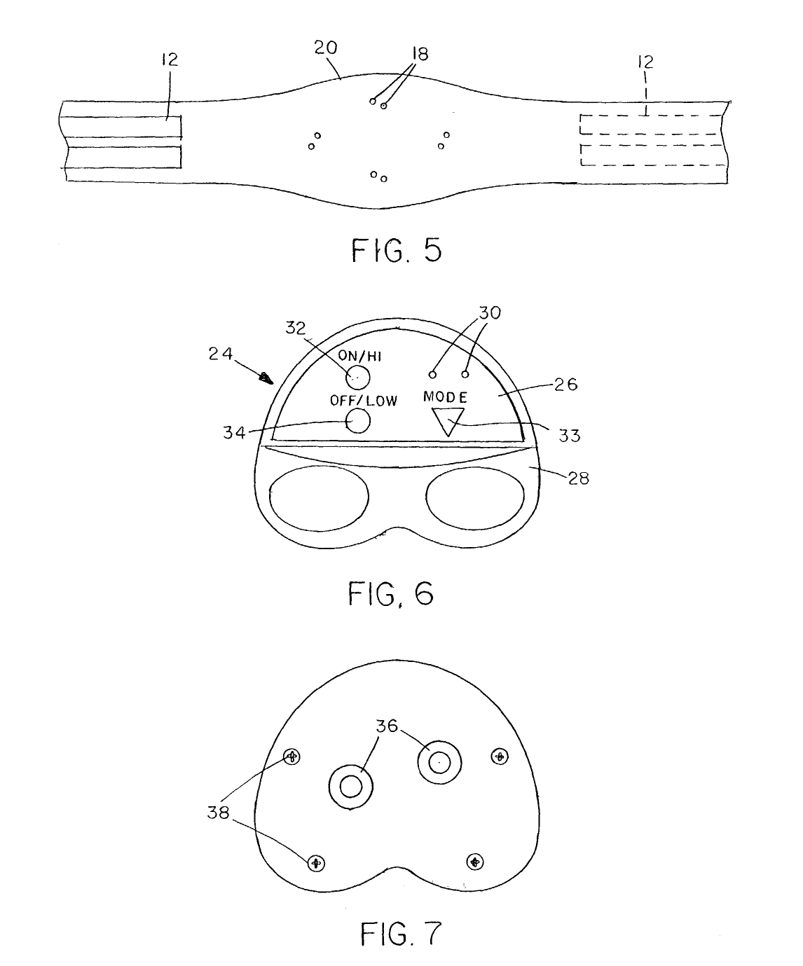

FIG. 5 is a top view of the belt 10 without the actuators 24. The top face of the belt contains at least one pair of male power connectors 18. An example of appropriate connectors that may be used are conventional snap connectors or any other like conductive connectors. Additionally, hook and loop fasteners 12 are contained on opposite ends and on the opposite faces of the belt 10 in order to allow for the belt 10 to be releasably attached to a desired body part. One skilled in the art will recognize that other types of releasable fasteners may be used but that hook and loop fasteners are an inexpensive yet efficient way to connect the ends of the belt to the user's body.

FIG. 6 is a top view of the actuator 24. The actuator 24 uses electrodes (located inside the actuator) to produce weak electrical impulses that cause selected muscles or muscle groups to contract and relax. Different muscles require varying levels of electrical impulses to reach full contraction. The time period between electrical impulses, i.e. when the muscles “relax” and rest between contractions, is critical to prevent muscle fatigue. Allowing sufficient time between contractions enable muscles to relax, reducing the possibility of muscle fatigue.

The top face of the actuator 24 comprises the control panel 26 and the battery cover 28. The control panel 26 has at least two LED's 30 indicating the status of the actuator (i.e. whether the actuator has been activated and what mode the actuator is operating in). Furthermore, the control panel 26 has a mode button 33 that allows the user to select the different modes. A first button 32 is utilized to activate the actuator 24, transmit electrical impulses and to increase the intensity of the electrical impulses. An initial press of the first button 32 causes the actuator 24 to activate or turn on. A second press of the first button 32 causes the actuator 24 to transmit electrical impulses. Additional presses of the first button 32 causes the intensity of the electrical impulses to increase. Each additional press of the first button 32 causes the intensity level of the electrical impulses to increase one level. The higher the intensity, the larger the contraction of the muscle.

Once the actuator 24 has been activated, an LED 30 on the control panel 26 will be lit up signaling that the actuator 24 is on. Once activated, a user can select from the different modes of the actuator 24. The mode selected is indicated by the LEDs 30. For example, the actuator 24 can be installed with two modes, a beginner mode and an advanced mode. The mode is selected by pressing the mode button 33. Pressing the mode button 33 once after the actuator 24 has been activated causes the beginner mode to be selected and pressing the mode button 33 a second time after the actuator 24 has been activated causes the advanced mode to be selected.

As previously mentioned, the LEDs 30 indicate the mode that has been selected by the user. If the beginner mode has been selected, one LED will be on and one LED will be off. If the advanced mode has been selected, both LEDs will be on. Furthermore, the beginner mode and the advanced mode are comprised of ten (10) intensity levels to offer a broad variation of different intensity levels, such as changing the length and pulse, of the electrical impulses for a flexible training program. A second button 34 is utilized to de-activate the actuator 24 and to lower the intensity level. Each time the second button 34 is pressed, the intensity level decreases by one until the actuator 24 has been de-activated or turned off. Additionally, contained adjacent to the control panel is a battery cover that is releasably engaged to the actuator. At least one battery is contained in the chamber defined between the top and bottom face of the actuator. This battery is a round, flat, 3V battery made of Alkaline or Lithium.

FIG. 7 is a bottom view of the actuator 24. The bottom face comprises at least one female actuator power connector 36 for releasable engagement to the male power connectors 18 contained on the top face of the belt 10. The power connectors 18 transmit the electrical impulses produced by the electrodes inside the actuator to the bottom ends of the power connectors 16 which in turn transmits them to the wiring pad causing the muscles to contract and relax. Furthermore, the actuator 24 is comprised of a front cover and a back cover. The electrodes producing the electrical impulses are contained between the front cover and the back cover. Screws 38 are utilized to keep the top face and bottom face togther and thus keeping the electrodes in place.

Although an exemplary embodiment of the invention has been described above by way of example only, it will be understood by those skilled in the field that modifications may be made to the disclosed embodiment without departing from the scope of the invention.

I claim:

1. An apparatus for applying electrical stimulation to a body of a user, the apparatus comprising:
a non-conducting material;
at least two pads, for transmitting electrical impulses from at least one actuator, located on the non-conducting material in an anatomic arrangement, corresponding to the symmetric muscle structure of a human body; and
at least one actuator releasably engaged on the belt for supplying the electrical impulses to the at least two pads.

2. The apparatus of claim 1, wherein the non-conducting material is a belt having a wide section in the middle and slender sections at each end of the belt.

3. The apparatus of claim 1, further comprising at least one pair of power connectors on the front side of the belt for attaching the at least one actuator.

4. The apparatus of claim 1, wherein the at least one actuator is comprised of a control panel for allowing different modes and intensity of electrical impulses to be controlled by the user.

5. The apparatus of claim 4, wherein power connectors are oriented on the top face of the belt in a diamond configuration and the at least one actuator is attached to the power connectors.

6. The apparatus of claim 5, wherein the at least two pads are arranged in accordance with the stomach muscle structure of the human body.

7. The apparatus of claim 4, further comprising a first button and a second button located on the control panel for selecting the mode and intensity of the electrical impulses.

8. A method for applying electrical stimulation to a body of a user, comprising the steps of:
securing a non-conductive material, having at least two pads, to the body of a user so the at least two pads are placed against muscles or a group of muscles;
placing at least one actuator on the non-conductive material to provide electrical impulses to muscles or groups of muscles causing the muscles to contract and relax; and
selecting a mode and intensity level of the electrical impulses to be applied to the muscles.

9. The method of claim 8, further comprising the steps of pressing a first button to increase the intensity of the electrical impulses and pressing a second button to decrease the intensity of the electrical impulses.

10. The method of claim 9, wherein the non-conducting material is a belt having a wide section in the middle and slender sections at each end of the belt.

11. The method of claim 10, wherein a gel is applied to the at least twp pads before the at least two pads are placed against the muscles or the group of muscles.
